(19) United States
(12) Patent Application Publication
(10) Pub. No.: US 2007/0207114 A1
(43) Pub. Date: Sep. 6, 2007

(54) Hair Growth Agent Composition

(75) Inventors: Seong-Lok Hwang, Chungcheongbuk-do (KR); Min-Ho Lee, Daejeon (KR); In-Byeong Yoon, Chungcheongbuk-do (KR); In-Ho Lee, Daejeon (KR); Su-Jin Park, Daejeon (KR); Seok-Seon Roh, Daejeon (KR)

Correspondence Address: VENABLE LLP, P.O. BOX 34385, WASHINGTON, DC, 20043-9998, US

(73) Assignee: LG HOUSEHOLD & HEALTH CARE LTD., SEOUL RPULBIC OF KOREA (KR)

(21) Appl. No.: 10/592,946

(22) PCT Filed: Mar. 16, 2004
(86) PCT No.: PCT/KR04/00561

§ 371 Date: Sep. 15, 2006

(51) Int. Cl. A61K 8/97; A61K 36/268

(52) U.S. Cl. 424/74; 424/773

(57) ABSTRACT

Disclosed herein is a composition for promoting hair growth. The composition comprises an Asiasari Radix extract as an active ingredient wherein the Asiasari Radix extract exhibits superior induction of the anagen phase of the hair growth cycle. The composition exhibits excellent hair loss prevention and hair growth promoting effects. In particular, when the composition further comprises an inhibitor of 5α-reductase activity and an activator of hair follicle cells, it exhibits better hair loss prevention and hair growth promoting effects.

### TECHNICAL FIELD

The present invention relates to a composition for preventing hair loss and promoting hair growth. More particularly, the present invention relates to a composition comprising an Asiasari Radix extract as an active ingredient wherein the Asiasari Radix extract exhibits superior induction of the anagen phase of the hair growth cycle by shortening the period taken in transfer from the telogen phase to the anagen phase of hair growth cycle, thereby rapidly regenerating hairs.

### BACKGROUND ART

Normal humans have about 100˜150 thousand hairs on the scalp. Each hair undergoes a hair growth cycle consisting of anagen, catagen and telogen phases, and is finally shed. The period of this cycle lasts for 3˜6 years. Humans lose 50˜100 hairs on average per day.

In general, alopecia is a disease caused when the proportion of hairs in the anagen phase is relatively small and that of hairs in the catagen and telogen phases is large in the hair growth cycle, resulting in abnormal hair loss.

As main causes of alopecia, numerous hypotheses have been discussed, for example, excessive secretion of male sex hormones, excessive sebum secretion, poor blood circulation, scalp dysfunction due to the presence of peroxides or bacteria, hereditary factors, aging, stress and the like. Accurate causes of alopecia still remain unknown. However, it is known that alopecia is associated with very complicated mechanisms.

In connection with the complicated causes of alopecia, drug products developed hitherto comprise various active ingredients having desired effects, e.g., blood circulation promotion, enhancement of hair root function, scalp moisturization, inhibition of dandruff, antioxidation, extension of the anagen phase of the hair growth cycle, inhibition of male sex hormone activity, etc. However, these drug products have been unsuccessful in terms of their effects, and they have been reported to cause some side effects.

In vivo assay using test animals and in vitro assay using hair follicle cells and tissue culture, etc., are mainly employed as experimental methods for preventing hair loss and promoting hair growth. Since methods for culturing dermal papilla cells and outer root sheath cells, which are main cells constituting hair follicles, have been developed in the 1980's, a number of investigations on the differentiation mechanisms in the hair follicles have been actively in progress. Culturing methods of hair follicle tissue developed until now have opened a possibility that they can be utilized in the investigations on the hair regeneration, hair growth and prevention of hair loss. Recently, isolation of a variety of growth factors and genes, investigations on mechanisms and drug screening associated with hair growth using biochemical and molecular biological experiments have been actively in progress. However, since these in vitro culturing techniques neglect interactions between hair follicle tissue cells, interactions between hair follicle tissue and hypodermal tissue surrounding the follicle tissue, and blood circulation, which are factors capable of directly or indirectly affecting hair growth, they are very limited in applicability. Accordingly, evaluations of effects and clinical trials using test animals are predominantly utilized to examine actual effects of drugs screened by a variety of biochemical techniques.

Although currently commercially available products for promoting hair growth or preventing hair loss have useful effects, e.g., induction of the anagen phase, extension of the anagen phase of the hair growth cycle, inhibition of 5α-reductase activity, blood circulation promotion, sterilization, inhibition of dandruff, moisturization, antioxidation, etc. They are unsatisfactory in the prevention of hair loss and the promotion of hair growth. This is because the causes of alopecia are very complicated and intricate. Conventional products have been developed focusing on only a few causes of hair loss or hair growth. In practice, preparations based on complicated and intricate mechanisms have not yet been developed.

Since male alopecia is dependent on male sex hormones, it is directly associated with the amount of male sex hormones. Thus, there have been a number of reports on methods for preventing and treating hair loss by inhibiting the activity of male sex hormones. Mechanisms of male sex hormones in alopecia will be briefly described below. Testosterone, one of the male sex hormones, is converted to dihydrotestosterone, an active male sex hormone, by the action of 5α-reductase. Dihydrotestosterone binds with a receptor to induce a protein causing hair loss, finally leading to alopecia. Due to the mechanism, excessive sebum secretion, acne or seborrheic dermatitis may be caused, and as a result, alopecia involving irritation of the scalp arises. In conclusion, male alopecia is due to excessive secretion of dihydrotestosterone by the action of 5α-reductase. Accordingly, it is believed that the inhibition of 5α-reductase activity will help in basic and effective prevention and treatment of male alopecia. From this viewpoint, for example, a therapeutic agent for male alopecia was developed using finasteride, which has been used as a therapeutic agent for the prostate gland. However, in connection with the use of finasteride, several side effects such as sexual dysfunction were reported. In addition, inhibition of 5c-reductase activity alone cannot achieve sufficient effects in the treatment of alopecia.

In view of the above-mentioned problems, the present inventors have screened several tens of plant extracts in order to identify the induction of the anagen phase of the hair growth cycle through animal experiments, and as a result, have found that an Asiasari Radix extract has superior induction of the anagen phase of the hair growth cycle. Specifically, the present inventors selected several tens of plant extracts useful for the prevention of hair loss and the promotion of hair growth while ensuring safety to humans without any toxicity, and conducted investigations to evaluate the induction of the anagen phase of the hair growth cycle. Finally, the present inventors have discovered that the Asiasari Radix extract exhibits superior induction of the anagen phase of the hair growth cycle by shortening the period taken in transfer from the telogen phase to the anagen phase in the hair growth cycle, thus accomplishing the present invention.

An application of an Asiasari Radix extract to hair-related products is disclosed in Korean Patent Laid-open No. 2001-0003366. According to this patent document, a solution of the Asiasari Radix extract in sesame oil is added to a mixture of Viticis Fructus, Cnidii Rhizoma, Angelicae Dahuricae Radix and Lysimachiae Foenumgraeci. However, the prior art has a poor solution to accomplish the technical task, and is very different from the present invention in terms of the mechanism. Further, Japanese Patent Laid-open No. Hei 10-265347 suggests the use of an Asiasari Radix extract for extending the anagen phase of the hair growth cycle. However, the mechanism described in the patent document is not associated with the induction effect of the Asiasari Radix extract on the anagen phase of the hair growth cycle disclosed in the present invention. Moreover, a combination of the Asiasari Radix extract and other active ingredients disclosed in the present invention exhibits better hair loss prevention and hair growth promoting effects.

Based on the fact that causes of alopecia are very complicated and intricate, the present inventors have earnestly conducted research to develop a composition for preventing hair loss and promoting hair growth from an optimum combination of active ingredients effective for each cause, and as a result, found that a composition comprising an Asiasari Radix extract, an ingredient inhibiting 5α-reductase activity and an ingredient activating hair follicle cells, as active ingredients, exhibits excellent hair loss prevention and hair growth promoting effects without any side effects.

Meanwhile, although there have been many reports on maintenance and extension of the anagen phase of the hair growth cycle through activating effects of hair follicle cells, extension effects of the anagen phase alone cannot achieve sufficient effects on hair growth promotion or hair loss prevention.

### DISCLOSURE OF THE INVENTION

Therefore, the present invention has been made in view of the above problems, and it is an object of the present invention is to provide a composition effective for the prevention of hair loss and the promotion of hair growth which can provide complex functions on many intricate mechanisms of alopecia from the viewpoint of induction of the anagen phase, inhibition of 5α-reductase activity and activation of hair follicle cells, which are considered as being effective functions in the promotion of hair growth and prevention of hair loss.

In order to accomplish the above object of the present invention, there is provided a composition for preventing hair loss and promoting hair growth which comprises an Asiasari Radix extract as an active ingredient.

The Asiasari Radix extract may be obtained by extracting Asiasari Radix with a lower alcohol, n-hexane or a mixture thereof. The Asiasari Radix extract thus obtained is preferably present in an amount of 0.01˜10% by weight based on the solid content, relative to the total weight of the composition according to the present invention.

If necessary, the composition of the present invention further comprises at least one selected from an ingredient inhibiting 5α-reductase activity (hereinafter, referred to as an “inhibitor of 5α-reductase activity”) and an ingredient activating hair follicle cells (hereinafter, referred to as an “activator of hair follicle cells”). It is preferred that each of the inhibitor of 5α-reductase activity and the activator of hair follicle cells is present in an amount of 0.001˜10% by weight, relative to the total weight of the composition.

The composition for promoting hair growth according to the present invention is very inventive over prior arts in that the Asiasari Radix extract exhibits hair growth promoting effects through a definite mechanism, i.e., induction of the anagen phase of the hair growth cycle, and the inhibitor of 5α-reductase activity and the activator hair follicle cells synergistically increase hair loss prevention and hair growth promoting effects.

Hereinafter, the composition of the present invention will be explained in more detail.

Considering the background of the present invention, the present inventors selected several tens of plant extracts useful for the prevention of hair loss and the promotion of hair growth while ensuring safety to humans without any toxicity, and conducted investigations to evaluate the induction of the anagen phase of the hair growth cycle. As a result, the present inventors have discovered that the Asiasari Radix extract exhibits superior induction of the anagen phase of the hair growth cycle. Accordingly, the present invention is based on this discovery. For better hair loss prevention and hair growth promoting effects, causes of alopecia must be investigated through a variety of mechanisms, and optimum combinations of active ingredients effective for each mechanism must be reviewed. For this purpose, the present inventors selected ingredients controlling male sex hormone activity, ingredients promoting blood circulation, ingredients promoting cell activity, ingredients having anti-inflammatory activity and the like among ingredients proven to be safe to humans, and combined these ingredients to search for an optimum combination having excellent hair loss prevention and hair growth promoting effects. Finally, the present inventors have found that a composition comprising an Asiasari Radix extract, an inhibitor of 5α-reductase activity and an activator of hair follicle cells, as active ingredients, exhibits excellent hair loss prevention and hair growth promoting effects. Further the present inventors have found an optimum combination of these active ingredients. The present invention is based on these findings.

Accordingly, the present invention provides a composition having excellent hair loss prevention and hair growth promoting effects which can provide complex functions on many causes of alopecia from the viewpoint of induction of the anagen phase, activation of hair follicle cells and inhibition of 5α-reductase activity, which are considered as being effective functions in the promotion of hair growth and prevention of hair loss.

Asiasari Radix used as a raw material in the composition of the present invention is a crude drug prepared from roots and rhizomes obtained by cutting the above-ground part of Asiasarum sieboldi and Asiasarum heterotropoides belonging to Aristolochiaceae. Asiasari Radix has a non-uniformly curved string shape, and includes 3˜5 mm thick, yellowish brown gnarled rhizomes and 5˜20 mm long, about 1 mm thick roots attached to the rhizomes. In the lengthwise direction of the roots, light brown shallow grooves are formed. At the upper part of the rhizome, leafstalks, peduncles or buds may exist. Fracture of the roots and the rhizomes is easy, and the fractured surface is yellowish white. Asiasari Radix has an inherent odor, and a hot and acrid taste. Representative major ingredients of Asiasari Radix include methyleugenol, cineol, safrole and 1-asarin. Asiasarum heterotropoides contains 1-sesamin, kakuol, asariein, saishinone, eucarvone and ingredients of other crude drugs belonging to the same genus.

In order to prepare the Asiasari Radix extract contained in the composition of the present invention, first, roots and rhizomes of Asiasari Radix are washed and finely ground, and then cold-immersed in an extraction solvent in an amount of about 10 times larger than the amount of the Asiasari Radix for 5 days. Thereafter, the resulting solution is filtered, concentrated and lyophilized to obtain a dry powder of the Asiasari Radix extract. The solvent used herein includes, but is not especially limited to, purified water; lower alcohols, e.g., methanol, ethanol, isopropyl alcohol n-butanol; polyhydric alcohols, e.g., glycerol, propylene glycol, 1,3-butyleneglycol; and hydrocarbon-based solvents, e.g., methylacetate, ethylacetate, benzene, n-hexane, diethyl ether, dichloromethane, etc. Among these solvents, lower alcohols such as methanol, ethanol and n-butanol, and n-hexane are preferred. These solvents may be used alone or in combination.

The Asiasari Radix extract thus obtained is preferably present in an amount of 0.01˜10% by weight (based on the solid content), relative to the total weight of the composition according to the present invention. When the content of the Asiasari Radix extract is less than 0.01% by weight, induction of the anagen phase of the hair growth cycle is unsatisfactory. On the other hand, when the content of the Asiasari Radix extract exceeds 10% by weight, an excessive amount of the Asiasari Radix extract does not contribute to further improvement in the induction of the anagen phase.

In addition to the Asiasari Radix extract, the composition of the present invention further comprises an inhibitor of 5α-reductase activity and an activator of hair follicle cells, as active ingredients, which are known to have a close relation to male alopecia. Accordingly, the composition of the present invention exhibits better hair loss prevention and hair growth promoting effects.

The inhibitor of 5α-reductase activity contained in the composition of the present invention is an ingredient for external application having inhibitory effects on 5α-reductase activity in human hair follicle cells and thus exhibiting hair loss prevention effects. One or more ingredients can be used alone or in combination as the inhibitor of 5α-reductase activity, so long as they do not pose safety risks to humans. Examples of suitable ingredients of the inhibitor of 5α-reductase activity include crude drug ingredients originating from plants, e.g., Sophorae Radix, Coicis Semen, Caryophylli Flos, Duckweed (Spirodela polyrhiza SEHLEIDEN), White birch (Betula platyphylla var. japonica), Galla Rhois, Angelicae Koreanae Radix, Rhizoma Ligustici, Rhei Rhizoma, Persicae Semen, Bombycis Corpus, Bletilla Striata, Arecae Semen, Perillae Folium, Curcumae longae Rhizoma, Paeoniae Radix (Paeonia albiflora, Paeonia lactiflora), Gleditis Sinensis, Gambir, Foeniculi Fructus, Polygalae Radix, Schizonepetae Herba, Pharbitidis Semen, Plantaginis Semen, Picrasma guassioides extracts. The above-mentioned crude drugs are commonly described in the Korean Pharmacopoeia or Korean Herbal Pharmacopoeia, and are commercially available in the market. These crude extracts are obtained by common solvent extraction process. The choice of extraction solvents is not especially limited. The content of the inhibitor of 5α-reductase activity is preferably in the range of 0.001˜10% by weight, and more preferably 0.01˜5% by weight, based on the total weight of the composition according to the present invention.

The activator of hair follicle cells contained in the composition of the present invention is an ingredient for external application having activating effects on human hair follicle cells and thus exhibiting hair loss prevention or hair growth promoting effects. One or more ingredients can be used alone or in combination as the activator of hair follicle cells, so long as they do not pose safety risks to humans. Specific examples of ingredients of the activator of hair follicle cells include hinokitiol, nicotinamide, vitamin B6, cepharanthin, biotin and pantothenic acid, and may be used alone or in combination. The content of the activator of hair follicle cells is preferably in the range of 0.001˜10% by weight, and more preferably 0.01˜5% by weight, based on the total weight of the composition according to the present invention.

That is, the composition of the present invention comprises the Asiasari Radix extract having superior induction of the anagen phase of the hair growth cycle, the inhibitor of 5α-reductase activity and the activator of hair follicle cells, as active ingredients. Preferred examples of the inhibitor of 5α-reductase activity include Sophorae Radix, Coicis Semen and Caryophylli Flos extracts. Preferred examples of the activator of hair follicle cells include hinokitiol and nicotinamide. In particular, the Asiasa Radix extract is present in an amount of 0.01˜10%, the inhibitor of 5α-reductase activity is present in an amount of 0.001˜10%, and the activator of hair follicle cells is present in an amount of 0.001˜10%, based on their solid content.

When these active ingredient having different mechanisms on hair loss prevention and hair growth promoting effects are used alone, their effects are insufficient. In contrast, the composition comprising the Asiasari Radix extract, the inhibitor of 5α-reductase activity and the activator of hair follicle cells exhibits excellent hair loss prevention and hair growth promoting effects. In one preferred embodiment of the present invention, it was confirmed through animal experiments and clinical tests in patients with alopecia that the composition exhibited excellent hair loss prevention and hair growth promoting effects.

In order to enhance the functions of the active ingredients and to impart additional functions to the composition so as to be suitable for patient's use, at least one auxiliary component selected from anti-dandruff agents, deep cleansing agents, refrigerants and humectants can be added to the composition of the present invention. In this case, the composition exhibits better hair loss prevention and hair growth promoting effects. Examples of auxiliary components, e.g., anti-dandruff agents, deep cleansing agents, refrigerants and humectants, include capsicum tincture, tocopherol acetate, benzyl nicotinate, piroctonolamine, salicylic acid, 1-menthol, etc. The amount of the auxiliary agents added is not particularly limited as long as they do not hinder the effects inherent to the active ingredients and can maintain the stability of formulations. Considering human safety and the stability of formulations, it is preferred that the amount of capsicum tincture and tocopherol acetate is in the range of 0.001˜10% by weight, the amount of benzyl nicotinate is in the range of 0.001˜0.1% by weight, the amount of piroctonolamine is in the range of 0.001˜1% by weight, and the amount of salicylic acid and 1-menthol is in the range of 0.01˜1% by weight.

The composition of the present invention may be formulated into ordinary dosage forms which can be applied to the alopecia afflicted scalp, for example, liquids, creams, pastes and solids. The addition of common additives to the composition of the present invention enables application to various hair-related products for promoting hair growth, for example, hair shampoos, conditioners, lotions, liquid tonics and the like. The hair-related products also include aerosols of the above-mentioned formations.

### BEST MODE FOR CARRYING OUT THE INVENTION

The present invention will now be described in more detail with reference to the following preferred examples. However, these examples are given for the purpose of illustration and are not to be construed as limiting the scope of the invention.

In the following test examples, C57BL/6 mice were used as animal models for evaluating induction of the anagen phase of the hair growth cycle. C57BL/6 mice with spontaneous alopecia have black body hair. Melanocytes of C57BL/6 mice are defined only in the hair follicles where the synthesis of melanin is synchronized with the hair growth cycle. Since the hair growth cycle in C57BL/6 mice can be judged by visually observing changes in skin color, C57BL/6 mice have been widely used as an animal model for investigating induction of the anagen phase of the hair growth cycle.

### PREPARATIVE EXAMPLE

### Preparation of Asiasari Radix Extract

An Asiasari Radix extract was prepared in accordance with the following procedure.

First, roots and rhizomes (500 g) of Asiasari Radix were pulverized and passed through a sieve (#10), and then about 5000 ml of ethanol (Korean Pharmacopoeia) was added thereto. The resulting solution was cold-immersed at room temperature for 5 days. The extract was filtered through Whatman #2 filter paper, and the obtained filtrate was then concentrated to dryness using a rotary evaporator under reduced pressure at 45° C. to obtain about 20 g (yield: about 4%) of an Asiasari Radix extract.

### TEST EXAMPLE 1

### Evaluation of Anajen Phase-Induction Effect of Asiasari Radix Extract

To evaluate induction effects of the Asiasari Radix extract on the anagen phase of the hair growth cycle, mice (C57BL/6, 42˜56 day old male) were used. C67BL/6 mouse is an animal widely used for investigating induction of the anagen phase of the hair growth cycle.

First, hairs around the backs of the mice were shaved using an electric razor. Then, the weight of an individual mouse was measured and all the mice were divided into a few groups of 12 each in such a manner that the weight distribution in each group was uniform. After the mice were allowed to adapt to their environments for 1 day, a control agent and a test agent were applied on the shaved area of the mice. At this time, 35% ethanol was used as the control agent, and ethanolic (35%) solutions at various concentrations of the solid Asiasari Radix extract obtained in Preparative Example above were used as the test agent. After the agents were applied on the back of each mouse in an amount of 100 μl once daily for 30 days, induction of the anagen phase of the hair growth cycle was observed. The induction of the anagen phase in each mouse was identified by calculating the ratios between the areas where new hairs were grown and the shaved areas, and averaging the obtained values. The anagen phase-induction effect according to varying concentrations of the Asiasari Radix extract were evaluated. The results are shown in Table 1 below.

TABLE 1

| | Concentration | | | | |
|---|---|---|---|---|---|
| | 0.001% | 0.01% | 1% | 10% | Control agent |
| Area ratio (%) | 24.8 | 42.4 | 68.8 | 72.4 | 12.0 |

As can be seen from Table 1, the Asiasari Radix extract exhibited superior induction of the anagen phase of the hair growth cycle in a concentration-dependent manner. The test agents at concentrations of lower than 0.01% exhibited better induction of the anagen phase than the control agent, but the effect was unsatisfactory. On the other hand, at concentrations of higher than 10%, an excessive amount of the Asiasari Radix extract did not show significant improvement in the induction of the anagen phase.

### EXAMPLES 1 TO 8 AND COMPARATIVE EXAMPLES 1 TO 6

Compositions comprising the Asiasari Radix extract, and at least one ingredient selected from Sophorae Radix extract, Coicis Semen extract, Caryophylli Flos extract, hinokitiol and nicotinamide as active ingredients were prepared so as to have the proportions indicated in Table 2 below.

TABLE 2

| Ingredients | | | | | | | | | Com. | Com. | Com. | Com. | Com. | Com. |
|---|---|---|---|---|---|---|---|---|---|---|---|---|---|---|
| (wt %) | Ex. 1 | Ex. 2 | Ex. 3 | Ex. 4 | Ex. 5 | Ex. 6 | Ex. 7 | Ex. 8 | Ex. 1 | Ex. 2 | Ex. 3 | Ex. 4 | Ex. 5 | Ex. 6 |
| Ethanol | 35 | 35 | 35 | 35 | 35 | 35 | 35 | 35 | 35 | 35 | 35 | 35 | 35 | 35 |
| Asiasari | 0.5 | 0.5 | 0.5 | 0.5 | 0.5 | — | 0.5 | 0.5 | — | — | — | — | — | — |
| Radix extract | | | | | | | | | | | | | | |
| Sophorae | — | 0.1 | — | — | — | 0.1 | 0.1 | 0.1 | 0.1 | — | — | — | — | — |
| Radix extract | | | | | | | | | | | | | | |
| Coix Semen | — | — | 0.1 | — | — | — | — | — | — | 0.1 | — | — | — | — |
| extract | | | | | | | | | | | | | | |
| Caryophylli | — | — | — | — | — | — | — | — | — | — | 0.1 | — | — | — |
| Flos Extract | | | | | | | | | | | | | | |
| Hinokitiol | — | — | — | 0.1 | | 0.1 | 0.1 | 0.1 | — | — | — | 0.1 | | — |
| Nicotinamide | — | — | — | — | 0.1 | — | — | — | — | — | — | — | 1.0 | — |
| Capsicum | — | — | — | — | — | — | — | 0.5 | — | — | — | — | — | 0.5 |
| tincture | | | | | | | | | | | | | | |
| Benzyl | — | — | — | — | — | — | — | 0.01 | — | — | — | — | — | 0.01 |
| nicotinate | | | | | | | | | | | | | | |
| Tocopherol | — | — | — | — | — | — | — | 0.1 | — | — | — | — | — | 0.1 |
| acetate | | | | | | | | | | | | | | |
| Piroctonol- | — | — | — | — | — | — | — | 0.1 | — | — | — | — | — | 0.1 |
| amine | | | | | | | | | | | | | | |
| Salicylic acid | — | — | — | — | — | — | — | 0.1 | — | — | — | — | — | 0.1 |
| L-menthol | — | — | — | — | — | — | — | 0.1 | — | — | — | — | — | 0.1 |
| Tween 60 | 0.5 | 0.5 | 0.5 | 0.5 | 0.5 | 0.5 | 0.5 | 0.5 | 0.5 | 0.5 | 0.5 | 0.5 | 0.5 | 0.5 |

| | |
|---|---|
| Purified | Added until the total weight reached 100% |
| water | |

### TEST EXAMPLE 2

### Evaluation of Anagen Phase-Induction of Through Animal Experiment

First, the induction effects of the compositions prepared in Examples 1 to 8 and Comparative Examples 1 to 6 on the anagen phase of the hair growth cycle was evaluated in the same manner as in Test Example 1 (evaluation of anagen phase-induction effect). The concentrated solid extract obtained in Preparative Example was used to prepare the compositions shown in Table 2. Table 3 shows evaluation results of induction effects of the compositions of Examples 1 to 8 and Comparative Examples 1 to 6 on the anagen phase of the hair growth cycle.

TABLE 3

| | Examples | | | | | | | | Comparative Examples | | | | | |
|---|---|---|---|---|---|---|---|---|---|---|---|---|---|---|
| | Example No. | | | | | | | | | | | | | |
| | 1 | 2 | 3 | 4 | 5 | 6 | 7 | 8 | 1 | 2 | 3 | 4 | 5 | 6 |
| Area | 64.0 | 85.8 | 77.8 | 82.2 | 75.2 | 66.8 | 98.8 | 99.8 | 43.4 | 38.6 | 39.2 | 56.0 | 41.8 | 34.2 |
| ratio | | | | | | | | | | | | | | |
| (%) | | | | | | | | | | | | | | |

As shown in Table 3, the compositions of Comparative Examples 1 to 6 comprising an inhibitor of 5α-reductase activity and an activator of hair follicle cells exhibited hair growth promoting effects, but the effects were unsatisfactory. The compositions comprising the Asiasari Radix extract and the Sophorae Radix extract or the Coicis Semen extract (Examples 2 and 3), and the compositions comprising the Asiasari Radix extract and hinokitiol or nicotinamide (Examples 4 and 5), exhibited better hair growth promoting effects than the compositions comprising one of these active ingredients. The compositions comprising the Asiasari Radix extract, the Sophorae Radix extract and hinokitiol (Examples 7 and 8) exhibited better hair growth promoting effects than the compositions comprising one or two of these active ingredients.

These results indicate that the combination of induction of the anagen phase by the Asiasari Radix extract, inhibition of 5α-reductase activity and activation of hair follicle cells can exhibit surprising synergic effects on hair growth promotion.

As is evident from these results, the Asiasari Radix extract, the Sophorae Radix extract and hinokitiol exhibited excellent hair growth promoting effects through induction of the anagen phase of the hair growth cycle, inhibition of 5α-reductase activity and activation of hair follicle cells, respectively, all of which are associated with hair loss prevention and hair growth promoting effects. In addition, each active ingredient exhibited inherent activity without affecting the activity of other active ingredients. Accordingly, it is expected that since the combination of the Asiasari Radix extract, the Sophorae Radix extract and hinokitiol in an appropriate ratio provides complex functions on many intricate mechanisms of alopecia, the composition of the present invention can exhibit better hair loss prevention and hair growth promoting effects than any one of these active ingredients alone.

Thereafter, liquid hair tonics were prepared from the composition comprising the Asiasari Radix extract, the inhibitor of 5α-reductase activity and the activator of hair follicle cells as active ingredients, along with auxiliary ingredients. Clinical tests were performed in patients with alopecia using the liquid hair tonics.

### TEST EXAMPLE 3

### Clinical Test Using Composition Comprising Asiasari Radix Extract, Sophorae Radix Extract and Hinokitiol

Using the compositions comprising the Asiasari Radix extract, the Sophorae Radix extract, hinokitiol and other auxiliary ingredients, clinical test was performed to identify the effects of the compositions in patients with alopecia.

In clinical tests, the composition of Example 7 comprising the Asiasari Radix extract, the Sophorae Radix extract and hinokitiol, the composition of Example 8 comprising the Asiasari Radix extract, the Sophorae Radix extract, hinokitiol and other auxiliary ingredients, and the composition of Comparative Example 6 comprising no active ingredients were used to evaluate hair loss prevention and hair growth promoting effects.

The compositions were applied to 15 patients (adult men and women aged 20-50) suffering from male alopecia and 15 patients (adult men and women aged 20-50) who on average lost 100 or more hairs per day. The application was carried out by dropping 2˜3 droplets (about 2 ml) per day, and continued over a minimum of 1 month to a maximum of 4 months according to their effects.

The evaluation was performed by counting the number of hairs lost per day upon washing and by visual observation. The results were classified into the following five 5 levels (Table 4) and scored. Final judgment on the effects was expressed as an average value of the scores. When the average value was 3.0 or higher, it was judged to be effective. The results are shown in Table 5 below.

TABLE 4

| | Number of hairs lost | |
|---|---|---|
| Level | upon washing | Visual observation |
| 5 | Considerable decrease | Significant improvement |
| 4 | Average decrease | Average improvement |
| 3 | Slight decrease | Slight improvement |
| 2 | No change | No change |
| 1 | Increase | Poor |

TABLE 5

| | Number of hairs lost | |
|---|---|---|
| Example No. | upon washing | Visual observation |
| Example 7 | 3.6 | 3.4 |
| Example 8 | 4.1 | 3.7 |
| Comparative Example 6 | 2.6 | 2.4 |

As can be seen from Table 5, the composition of Comparative Example 6 exhibited slight improvements, but the effectiveness was lacking; whereas the composition of Example 7 comprising all active ingredients exhibited slight improvements or more, and the composition of Example 8 comprising further other auxiliary ingredients exhibited average improvements or more. Accordingly, it was confirmed that the compositions of Examples 7 and 8 were excellent in hair loss prevention and hair growth promoting effects. Moreover, since no side effects in test subjects were observed during the clinical test, the composition of the present invention was proved to be safe.

Consequently, it was found through animal experiments that the Asiasari Radix extract has superior induction of the anagen phase of the hair growth cycle. It was also found that the combination of the Asiasari Radix extract, the inhibitor of 5α-reductase activity and the activator of hair follicle cells exhibits better hair growth promoting effects than when using only one of these active ingredients. Further, the composition comprising three active ingredients was confirmed through animal experiments and clinical tests in patients with alopecia to exhibit excellent hair loss prevention and hair growth promoting effects. Accordingly, it is expected that since the combination of these active ingredients in an appropriate ratio provides complex functions on many intricate mechanisms of alopecia, the composition of the present invention can exhibit better hair loss prevention and hair growth promoting effects than any one of these active ingredients alone.

### INDUSTRIAL APPLICABILITY

As apparent from the above description, the present invention provides a composition comprising an Asiasari Radix extract as an active ingredient for promoting hair growth wherein the Asiasari Radix extract exhibits superior induction of the anagen phase of the hair growth cycle. The composition of the present invention can exhibit excellent hair loss prevention and hair growth promoting effects without any toxicity. In particular, when the composition further comprises an inhibitor of 5α-reductase activity and an activator of hair follicle cells, it is very safe and exhibits better hair loss prevention and hair growth promoting effects.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for preventing hair loss and promoting hair growth, comprising an Asiasari Radix extract as an active ingredient.

2. The composition according to claim 1, wherein the Asiasari Radix extract is in an amount of 0.01˜10% by weight based on the solid content, relative to the total weight of the composition.

3. The composition according to claim 1, wherein the Asiasari Radix extract is obtained by extracting Asiasari Radix with a lower alcohol, n-hexane or a mixture thereof.

4. The composition according to claim 2, wherein the Asiasari Radix extract is obtained by extracting Asiasari Radix with a lower alcohol, n-hexane or a mixture thereof.

5. The composition according to claim 1, further comprising at least one ingredient selected from an inhibitor of 5α-reductase activity and an activator of hair follicle cells.

6. The composition according to claim 5, wherein the inhibitor of 5α-reductase activity is at least one selected from Sophorae Radix extract, Coicis Semen extract and Caryophylli Flos extract; and the activator of hair follicle cells is at least one selected from hinokitiol and nicotinamide.

7. The composition according to claim 5, wherein the inhibitor of 5α-reductase activity is in an amount of 0.001˜10% by weight and the activator of hair follicle cells is in an amount of 0.001˜10% by weight, based on the total weight of the composition.

8. The composition according to claim 1, further comprising at least one selected from capsicum tincture, tocopherol acetate, benzyl nicotinate, piroctonolamine, salicylic acid and 1-menthol.

9. The composition according to claim 5, further comprising at least one selected from capsicum tincture, tocopherol acetate, benzyl nicotinate, piroctonolamine, salicylic acid and 1-menthol.

10. A topical formulation for transdermal application, comprising the composition according to claim 1.
